(19) United States
(12) Patent Application Publication
(10) Pub. No.: US 2006/0177879 A1
(43) Pub. Date: Aug. 10, 2006

(54) Magnetic nanoparticles and method of fabrication

(76) Inventors: Eric L Mayes, Bristol (GB); Barnaby Warne, Bristol (GB); Kim K.W Wong, Bristol (GB)

Correspondence Address: KIRKPATRICK & LOCKHART NICHOLSON GRAHAM LLP, STATE STREET FINANCIAL CENTER, ONE LINCOLN STREET, BOSTON, MA, 02111-2950, US

(21) Appl. No.: 10/530,109

(22) PCT Filed: Oct. 6, 2003
(86) PCT No.: PCT/GB03/04298

§ 371 Date: Mar. 28, 2006

(30) Foreign Application Priority Data
Oct. 4, 2002 (GB) 0223127.2

(51) Int. Cl. G01N 33/53; H01F 1/04; H01F 1/14

(52) U.S. Cl. 435/7.5; 252/62.55; 977/900

(57) ABSTRACT

A method for making a composition of magnetic nanoparticles. The method includes the step of forming the magnetic nanoparticles, each within a protein template, wherein a liquid composition of said protein templates or subunits thereof is subjected to a microporous membrane filtration step prior to formation of said magnetic nanoparticles.

The present invention relates to a process for the preparation of a stable composition of magnetic nanoparticles in a liquid and to a process for producing such stable compositions. The compositions of the present invention have a variety of end uses, but in particular are useful in the production of magnetic recording media.

### BACKGROUND OF THE INVENTION

Nanoparticulate material is becoming widely applied to many areas of technology, for example data storage media (WO98/22942), biomedical applications such as diagnostics and therapeutics (U.S. Pat. No. 5,491,219), bio-detection systems, cytomagnetometry, heat transfer media, sealants, damping agents, inks, transduction and pressure sensors (WO01/39217). It is known that such nanoparticles may be encapsulated, at least during their synthesis. The encapsulating material may be retained or removed on completion of the synthesis of the nanoparticle.

A common problem encountered in the art of producing magnetic nanopartioles is the tendency of the particles to aggregate, thereby making their application problematic (Kumar K 1997, J. Liq. Chromat. & Related Tech. 20 (20) pages 3351-3364). Ideally, once produced, a nanoparticulate composition should have a high degree of stability with a low degree of aggregation of the nanoparticles.

Whilst it might be expected that one could obtain an enriched composition of magnetic nanoparticles by exploiting their magnetic properties, it is commonly found that once magnetic nanoparticles are subject to magnetic fractionation they become irreversibly aggregated.

Various approaches such as sonication, the use of dispersants and the modification of surface charge have been employed to try to overcome aggregation problems of magnetic nanoparticles (Rittner M, Business Communications Co. Inc. GB-201A-C December 2001 Opportunities in Nanostructured Materials) often with limited success.

Previous studies considering the prevention of aggregation of ferritin-encapsulated nanoparticles have largely focussed on size-exclusion chromatography and gel filtration (Kumar K 1997 ibid; Hainfeld J F, 1992 PNAS 89 pages 11064-11068) as a means to overcome aggregation. The outcome of the methodologies described in these reports was not favourable in terms of recovering particles with a low tendency to aggregate.

We have devised an improved method of producing stable compositions of magnetic nanoparticles. In accordance with the invention, a liquid composition comprising the encapsulated magnetic nanoparticles (or a liquid composition of the encapsulating material, when the encapsulating material is a protein template which is intended for the formation of the magnetic nanoparticles) is subjected to a membrane filtration step. This improves the stability of the resulting magnetic nanoparticles, in particular their resistance to aggregation. Further, a combination of magnetic fractionation and filtration of magnetic nanoparticles can yield populations of highly occupied, encapsulated magnetic nanoparticles that do not aggregate for considerable periods. A composition of highly occupied magnetic nanoparticles may for example be a composition within which the majority of the encapsulating particles contain at least a small magnetic nanoparticle. In addition, in such compositions, a fraction of the encapsulating particles may be substantially filled by magnetic nanoparticles. Alternatively it may be a composition within which a fraction of the encapsulating particles are substantially filled by larger magnetic nanoparticles.

### SUMMARY OF THE INVENTION

In accordance with a first process aspect of the present invention, there is provided a method for making a composition of magnetic nanoparticles which includes the step of forming said magnetic nanoparticles each within a protein template, wherein a liquid composition of said protein template or subunits thereof is subjected to a microporous membrane filtration step prior to formation of said magnetic nanoparticles.

In accordance with a second process aspect of the present invention, there is provided a method for treating a liquid composition of magnetic nanoparticles, each formed within a macromolecular template, wherein said method includes the step of subjecting said composition to a microporous membrane filtration step.

It has been found that the methods according to the first and second aspects of the invention provide highly stable compositions of encapsulated magnetic nanoparticles having a high resistance to aggregation. In one embodiment of the invention we have been able to generate compositions of magnetic nanoparticles which do not show signs of aggregation for at least six months.

In accordance with a third aspect of the present invention, there is provided a stable composition of magnetic nanoparticles wherein each nanoparticle is encapsulated by an encapsulating material, wherein at least 70% by weight of the nanoparticles are not in an agglomerated form and wherein the composition comprises no more than 30% free encapsulating material, based on the total weight of the encapsulating material in the composition. In a preferred embodiment of the third aspect of the invention, the composition comprises no more than 10% free encapsulating material, based on the total weight of the encapsulating material in the composition. Such compositions are obtainable in accordance with the process aspects of the present invention. In this aspect of the invention, it is preferred that over 80% by weight of the particles are not agglomerated, more preferably at least 90% by weight. The degree to which the particles are aggregated can be assessed using Transmission Electron Microscopy (TEM) (Jeol 2010; http://www.jeoleuro.com) or Atomic Force Microscopy (“Dimension”, Digital Instruments; www.di.com).

By “agglomerated form”, we mean encapsulated particles which are present in clumps of particles and not as discrete particles which are spatially separated from each other in the composition.

By “free encapsulating material”, we mean encapsulating material which does not contain a core magnetic nanoparticle, or which may be regarded as substantially unmineralised.

### FIGURES

FIG. 1 shows transmission electron micrographs (JEOL 2010) of the cobalt-platinum nanoparticles within apoferritins both (a) before and (b) after magnetic separation.

### DETAILED DESCRIPTION OF THE INVENTION

In each of the first two process aspects of the present invention, the magnetic nanoparticles and encapsulating particles comprise part of a liquid composition. The liquid composition may be regarded as a “solution” in the sense that the components thereof are generally regarded as being solubilized, although such solutions can also be regarded as colloidal suspensions. The predominant component of the liquid composition is preferably water, although a percentage of one or more water-miscible solvents may also be present such as tetrahydrofuran or ethanol. For example tetrahydrofuran or other water miscible solvents may be present in a total amount of up to 50% by weight. The percentage of water-miscible solvents in the liquid composition is preferably less than 25% by weight, more preferably less than 10% by weight.

In each of the two process aspects of the invention, the liquid composition is subjected to a membrane filtration step. Membrane filters are well known structures which are distinguished from non-membrane filters by the fact that membranes have a structure which is monolithic, i.e. the solid structure is permanently bonded forming a continuous solid phase. In contrast, non-membrane filters are formed by fibres held in place by mechanical entanglement or other surface forces. Membrane filters can be made with narrow pore size distribution with very small pores when necessary. The microporous membranes used in the present invention have pores approximately in the range 0.02-10 μm, preferably less than 1 μm and most preferably less than 0.5 μm; specific examples of pore sizes which may be used in the present invention are pores of 0.2 μm and pores of 0.1 μm. The microporous filter used in the invention may be made from various materials, including polymers, metals, ceramics, glass and carbon. Typically the membrane will be formed of a polymeric material known in the art to be used in membrane filtration, such as for example polysulphones, polyethersulphones (PES), polyacrylates, polyvinylidenes, for example polyvinylidene fluoride (PVDF), polytetrafluoroethylene (PTFE), cellulose, cellulose esters or co-polymers thereof. Preferably where the encapsulating material is a protein, the membrane will be selected to comprise a low protein-binding material such as a polyethersulphone or a polyvinylidene. Such microporous filters are available from Millipore Corporation (Bedford, Mass.).

The membrane filter may be a membrane disc, although other forms of membrane filters are usable in the present invention.

Significantly, we have found that to achieve the production of stable magnetic nanoparticle compositions filter pore size can be several orders of magnitude greater than that of the nanoparticulate material. For example, in a preferred embodiment of the invention the encapsulating material, apoferritin, has an approximate diameter of 12 nm. We have found that stable preparations of ferritin encapsulated magnetic nanoparticles which are resistant to aggregation can be achieved using 0.2 μm and 0.1 μm filters.

Normally, the magnetic nanoparticles of the invention will have all of their dimensions in the nano size range, typically at least 1 nm and no greater than 100 nm, preferably no greater than 50 nm and more preferably no greater than 20 nm. Preferred magnetic nanoparticles of the invention are substantially spheroidal having a diameter in the range 1-100 nm. However the present invention also extends to magnetic particles which have one dimension which is not within the nanosize range, as for example, the particles formed using microtubules which are tubular proteins, formed from αβ-tubulins, and have an outer diameter of about 25 nm and a length of several micrometres.

In the first process aspect of the present invention, a liquid composition of the protein template or subunits is subjected to a microporous membrane filtration step prior to the formation of said magnetic nanoparticles. Thus, in this embodiment, a liquid composition of the protein template is first prepared, normally an aqueous solution, which is then subjected to the microporous membrane filtration step. In this step, the composition is introduced to one side of the filter and filtered through the membrane. In this embodiment, the protein in the composition is preferably present at a concentration in the range from 10-50 mg/ml. In an embodiment, the pH of the composition is preferably in the range from 5-7. Preferably, the composition is subjected to an applied positive pressure during the filtration step. For example the applied pressure may be greater than 1 psi, for instance greater than 5 psi. Normally, the pressure will be less than 20 psi, for instance less than 15 psi. The filtrate, which comprises a composition of the protein template (or a subunit thereof) is then recovered, for use in the encapsulation of magnetic nanoparticles in a manner which is known per se (see WO 98/22942).

In the second process aspect of the invention, a liquid composition of the magnetic nanoparticles, each formed within a macromolecular template is subjected to the microporous membrane filtration step. In this process aspect of the invention, magnetic nanoparticles are first formed within a macromolecular template in a manner which is known per se (see for example WO 98/22942). Whilst in this aspect the preferred macromolecular template is a protein template, this is not essential and other macromolecular materials may be used for the formation of the magnetic nanoparticles. A composition of the magnetic nanoparticle, preferably an aqueous solution although other solvents such as alcohols or alkanes may be used in some embodiments, is then subjected to the microporous membrane filtration step. In this filtration step, as with the first process aspect of the invention, the composition is introduced to one side of the filter and filtered through the membrane. In this embodiment of the invention, the magnetic nanoparticles are preferably present in the composition in a concentration in the range from 0.1-20 mg/ml. In an embodiment, the pH of the composition is preferably in the range from 7-8.5. Preferably, the composition is subjected to an applied positive pressure during the filtration step. For example the applied pressure may be greater than 1 psi, for instance greater than 5 psi. Normally, the pressure will be less than 20 psi, for instance less than 15 psi. The filtrate, which comprises a composition of the encapsulated magnetic nanoparticles is then recovered.

The encapsulating material used in the second process aspect of the present invention and which encapsulates the magnetic nanoparticle in the third (product) aspect of the present invention should be capable of accommodating or at least partially accommodating the magnetic nanoparticle, and may therefore comprise a suitable cavity capable of containing the particle; such a cavity will normally be fully enclosed within the encapsulating material. Alternatively, the encapsulating material may include a suitable opening which is not fully surrounded, but which nevertheless is capable of receiving and supporting the magnetic particle; for example, the opening may be that defined by an annulus in the macromolecule.

The encapsulating shell may comprise organic material or inorganic material such as siloxanes, silanes or derivatives thereof. The encapsulating material may comprise a single particle or a number of particles which act together to accommodate the core magnetic nanoparticle.

In a preferred embodiment, the encapsulating material may be an organic macromolecule by which we mean a molecule, or assembly of molecules, and may have a molecular weight of up 1500 kD, typically less than 500 kD. Such organic macromolecular molecules may be surfactants, polymers or proteins. Suitable proteins include flagellar L-P rings, microtubules which are tubular proteins, formed from (αβ-tubulins, and have an outer diameter of about 25 nm and a length of several micrometres, bacteriophages, chaperoninins such as the bacterial GroEL and GroES, DPS and virus capsids. For example, DPS, is a ferritin homologue, dodecamer DNA protection protein comprising a hollow core and pores in the three-fold axis. Flagellar LP rings are ring-shaped structures having an inner diameter of approximately 13 nm and outer diameter of approximately 20 nm. They can be induced to pack into well-ordered arrays extending over several microns, approximately 13 nm thick. At more dilute concentrations, dimers can form that are 26 nm thick.

In a highly preferred embodiment, the encapsulating material is a member of the ferritin family. The present invention most preferably makes use of the iron storage protein, ferritin, whose internal cavity is used to produce nanoscale magnetic particles. Ferritin has a molecular weight of 450 kD. Ferritin is utilised in iron metabolism throughout living species and its structure is highly conserved among them. It consists of 24 subunits which self-assemble to provide a hollow shell roughly 12 nm in outer diameter. It has an 8 nm diameter cavity which normally stores 4500 iron(III) atoms in the form of paramagnetic ferrihydrite. However, this ferrihydrite can be removed (a ferritin devoid of ferrihydrite is termed “apoferritin”) and other materials may be incorporated. The subunits in ferritin pack tightly; however there are channels into the cavity at the 3-fold and 4-fold axes. The presently preferred macromolecule for use in the invention is the apoferritin protein, which has a cavity of the order of 8 nm in diameter. The magnetic nanoparticle to be accommodated within this protein will have a diameter up to about 15 nm in diameter, as the protein can stretch to accommodate a larger particle than one 8 nm in diameter.

Ferritin can be found naturally in vertebrates, invertebrates, plants, fungi, yeasts, bacteria. It can also be produced synthetically through recombinant techniques. Such synthetic forms may be identical to the natural forms, although it is also possible to synthesise mutant forms which will still retain the essential characteristic of being able to accommodate a particle within their internal cavity. The use of all such natural and synthetic forms of ferritin is contemplated within the present invention.

Ferritin may be converted to apoferritin by dialysis against a buffered sodium acetate solution under a nitrogen flow. Reductive chelation using, for example, thioglycolic acid may be used to remove the ferrihydrite core. This may be followed by repeated dialysis against a sodium chloride solution to completely remove the reduced ferrihydrite core from solution.

The description in the preceding paragraphs which pertain to embodiments in which the encapsulating material is a protein (or a subunit thereof) are pertinent to the first process aspect of the present invention. Thus, suitable proteins for use in the first aspect of the present invention include flagellar L-P rings, microtubules which are tubular proteins, formed from αβ-tubulins, and have an outer diameter of about 25 nm and a length of several micrometres, bacteriophages, chaperoninins, DPS such as GroEL and virus capsids. The preferred protein template material is a member of the ferritin family, whose internal cavity is used to produce nanoscale magnetic particles.

The magnetic nanoparticles of the present invention, as previously mentioned, have a diameter (or largest diameter in the case of non-spheroidal particles) not greater than 100 nm. Preferably the diameter is not greater than 50 nm, more preferably it is 20 nm or less. This dimension is determined, at least in part by the size of the encapsulating material. Where the encapsulating material is apoferritin which has a cavity of about 8 nm in the relaxed state, the core magnetic nanoparticle (that is to say the core material excluding the encapsulating material) may have a diameter up to about 15 nm in diameter, as the protein can stretch to accommodate a larger particle than one 8 nm in diameter.

The magnetic core particle may be either ferrn or ferro-magnetic metals such as cobalt, iron, or nickel; a metal alloy, rare earth and transition metal alloy, M-type or spinet ferrite. The metal or metal alloy may contain one or more of the following: aluminium, barium, bismuth, cerium, chromium, cobalt, copper, dysprosium, erbium, europium, gadolinium, holmium, iron, lanthanum, lutetium, manganese, molybdenum, neodymium, nickel, niobium, palladium, platinum, praseodymium, promethium, samarium, strontium, terbium, thulium, titanium, vanadium, ytterbium, and yttrium or a mixture thereof.

Preferably said nanoparticles comprise a binary alloy or ternary alloy such as cobalt-nickel, iron-platinum, cobalt-palladium, iron-palladium, samarium-cobalt, dysprosium-iron-turbide or neodymium-iron boride, iron-cobalt-platinum, cobalt-nickel platinum, or cobalt-nickel-chromium. Preferably, said nanoparticles comprise cobalt or platinum or alloys thereof. More preferably still, said nanoparticles comprise an alloy of cobalt and platinum.

The magnetic nanoparticles may be prepared by a process in which a solution of the encapsulating material such as an organic macromolecule, typically in an aqueous medium, is combined with a source of ions of the appropriate metal or metals to comprise or consist the core magnetic nanoparticle. In the process of the present invention, it is preferred that the source of metal ions be added incrementally to the source of the encapsulating material. For example the cation and anion sources may be added in sufficient amounts to provide more than 1 atom of the cation and anion sources per encapsulating particle per iteration, preferably more than 20 atoms of the cation and anion sources per encapsulating particle per iteration. The cation and anion sources may be added in sufficient amounts to provide fewer than 200 atoms of the cation and anion sources per encapsulating particle per iteration, preferably fewer than 100 atoms of the cation and anion sources per encapsulating particle per iteration. In a preferred embodiment of the invention the cation and anion sources may be added in sufficient amounts to provide about 50 atoms of the cation and anion sources per encapsulating particle per iteration. These low concentrations may be achieved by successive dilutions of solutions containing the cation and anion sources.

In one embodiment of the invention, it is preferred that the source of metal ions be a salt of the metal or metals, for example tetrachloroammoniumplatinate, comprising the magnetic nanoparticle.

Alternatively, but presently less preferred, the source of metal ions may be present in a composition to which a source of organic macromolecule is added.

The mixture of organic macromolecules and metal ions may be agitated to ensure homogenisation. Where the nanoparticle is to comprise the elemental metal, a reduction is effected on the composition whereby a nanoscale metal particle forms within the organic macromolecule cavity. This reduction preferably takes place under an inert atmosphere to protect the metal particles from oxidation, which would reduce their magnetic properties. The reduction/oxidation step may be repeated between additions of metal ions (which may be the same or different in each cycle) to build up the nanoparticles.

The reaction mixture may be formed at a temperature below the preferred temperature at which the magnetic nanoparticles are allowed to form and then raised to that temperature. Alternatively, the source of encapsulating material to which the source(s) of metal ions is to be added may be held at a temperature of at least 24° C. and the metal ion source(s) added thereto.

Proteins can generally withstand temperatures of up to 70° C. before they lose their tertiary structure. Thus in embodiments wherein the encapsulating material is a protein, the temperature of the reaction may range up to about 70° C. For these embodiments, the reaction temperature is preferably maintained in the range from about 25° C. to about 60° C., more preferably in the range from about 35° C. to about 50° C. Alternatively, the reaction temperature may be maintained in the range from about 50° C. to about 60° C., for example about 55° C. In another embodiment, the reaction temperature may be maintained in the range from about 60° C. to about 70° C., for example about 65° C.

In one embodiment of the invention, the aqueous medium is maintained at alkaline pH during the formation of the magnetic core particles within the macromolecular templates. The pH is preferably maintained in the range from 7.5-8.5. This may be achieved by the use of a buffer solution. Suitable solutions will vary depending on the encapsulating agent used.

In one embodiment of the process aspects of the invention, the method further includes a magnetic fractionation step of the encapsulated magnetic nanoparticles. This involves passing the composition through a retarding medium under gravity or by the exertion of a positive pressure whilst subjecting it to a magnetic field, such that the particles within the composition are spatially separated according to their magnetic properties; thus providing a means of obtaining a concentrated composition of particles of similar magnetic properties. Since the magnetic properties of the magnetic nanoparticles, whether encapsulated or not, will be determined by the size of the core magnetic nanoparticle, this method also provides a means for obtaining a composition wherein the core particles have a high degree of monodispersity i.e. the degree to which the size of the individual magnetic nanoparticles varies within a composition of the invention is low. This variation, measured in terms of the largest nano-sized dimension, should normally be less than 20%, preferably less than 10% and most preferably less than 5%. For compositions in which the average size is relatively large, e.g. about 50 nm, it is preferred that the variation is at the lower end of the above ranges, whilst for relatively small particles, e.g. about 10 nm, the variation may be at the upper end of the above ranges. The sizes of the particles in accordance with the present invention can be measured using for example Transmission electron microscopy (TEM).

The retarding medium may comprise steel, for example type IV 20 L, or another suitable soft-magnetic material in the form of a powder, beads or other form known in the art. It is preferred that the retarding medium comprise a material which does not react chemically with the magnetic nanoparticle composition in such a way as to damage or alter its structure, although it may be such that the magnetic nanoparticles have some form of attractive interaction during their passage through the fractionating device.

One skilled-in-the-art would appreciate that many means for magnetic fractionation are available such as magnetic wire, magnetic powder chromatography and field-flow fractionation techniques. In a preferred embodiment of the invention, the composition is passed through columns comprising magnetic powder at flow rates ranging from 0.2-10 ml/min. Magnetic fractionation also provides the advantage of enabling the fluid medium in which the nanoparticles are suspended to be exchanged.

In the case of the second process aspect of the invention in which the formed encapsulated magnetic nanoparticles are subjected to a membrane filtration step, this filtration step preferably occurs after the magnetic fractionation step.

In one embodiment of the invention the encapsulating shell provides a surface which can be functionalised for example with biotin/avidin to promote the attachment of biological ligands such as antibodies or fragments thereof. A variety of ligands such as antibodies or derivatives thereof, receptor molecules, opsonins etc. may be attached to the surface of the protein capsule. Further, a variety of protocols are available for the conjugation of binding moieties to the surface of the protein (Wong S. S. 1993 “Chemistry of protein conjugation and cross-linking” CRC Press) and, in particular, biotinylation and avidinylation of ferritin have been described (Li M. et. al. 1999 Chem. Mater., 11 pages 23-26; Bayer E. A. et. al. 1976 J. Histochem. & Cytochem., 24 (8) pages 933-939).

In an alterative embodiment of the invention the exterior aspect of the shell may be functionalised with, for example, a metal binding ligand to enable the medium to be used in applications for removing metal contaminants from materials such as waste materials.

In some embodiments, the encapsulating shell may be removed to leave the magnetic nanoparticle without a coating. For example, where the coating is a protein, this may be denatured through, for example enzymatic degradation or pH denaturation. In particular, the protein may be digested using proteases or denatured by adjusting the pH of the composition to a value outside the range at which the protein is stable, for example below about pH4.0 or above about pH9.0. The denatured protein material may then be removed by, for example, dialysis or centrifugation. In a preferred embodiment, the protein is denatured by adjusting the pH of the composition to below about 4.0.

In other embodiments, the shell may be treated to leave a residue surrounding the nanoparticle core, for example the macromolecular shell may be carbonised by isolating the encapsulated particles and subjecting them to an elevated temperature, for example of the order of 300° C., before re-suspending them in the desired carrier liquid. Alternatively, laser pyrolysis may be used if it is desired to carbonise the particles in composition.

The invention is now illustrated by reference to the following non-limiting examples:

### EXAMPLES

### Example 1

### Apoferritin Production

This example illustrates the preparation of apoferritin from horse spleen ferritin. Apoferritin was prepared from cadmium-free native horse spleen ferritin by dialysis (molecular weight cut-off of 10-14 kD) against sodium acetate solution (0.2 M) buffered at pH 5.5 under a nitrogen flow with reductive chelation using thioglycolic acid (0.3 M) to remove the ferrihydrite core. This was followed by repeated dialysis against sodium chloride solution (0.15 M) to completely remove the reduced ferrihydrite core from solution.

### Example 2

### Synthesis of Cobalt/Platinum Nanoparticles within Apoferritins

Apoferritin was dispersed in either 0.05M 4-(2-hydroxyethyl)-1-piperazineethane-sulfonic acid (HEPES) buffer, buffered to pH 7.5-8.5 or 0.25M AMPSO buffered to pH7-5.8.5. Aliquots of 0.1M cobalt (II) acetate solution and 0.1M ammonium tetrachloroplatinate (II) solution were then added and the mixture stirred at a temperature between 35 and 50° C. This was followed by reduction using sodium borohydride. A number of metal salt additions and subsequent reductions were performed to obtain apoferritin in which the cores were substantially occupied by Co/Pt crystals.

### Example 3

### Synthesis of Magnetite Particles within Apoferritins

Apo-ferritin was dispersed in 50 mM 3-([1,1-dimethyl-2-hydroxyethyl]amino)-2-hydroxypropane sulphonic acid (AMPSO) buffered, adjusted to pH 8.5 and the temperature maintained at between 40-70° C. Aliquots from solutions of ammonium iron (II) sulphate (25 mM) and trimethylamine-N-oxide (25 mM) were added incrementally to the apo-ferritin solution. The aliquot of iron (II) added was equivalent to 100 atoms per apo-ferritin molecule. The increment interval for the addition of aliquots was approximately 15 minutes. Additions were made until the apo-ferritin cores were substantially occupied by magnetite cores. The solution was then dialysed against water and filtered through 0.2 um filter before concentrating or using as prepared.

### Example 4

### Magnetic Separation of Magnetic Nanoparticles

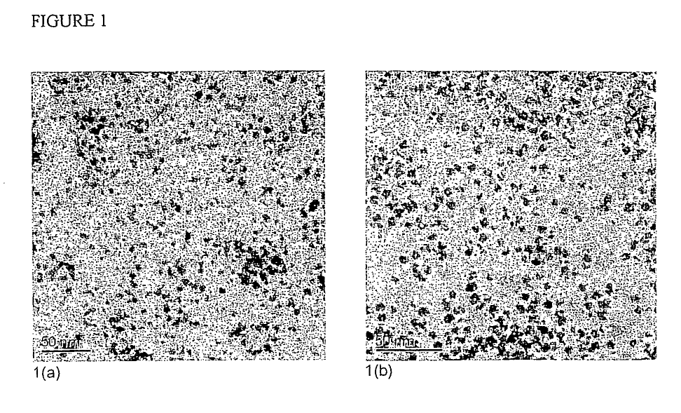

Magnetic separation was performed using glass columns containing steel powder columns. Two permanent magnets comprising neodymium iron boride were positioned either side of a section of the column and the magnetic nanoparticulate matter, for example the cobalt platinum-apoferritins particles illustrated in Example 2, were passed through the column. The column was subsequently washed with a deaerated solution of 0.25% (w/v) hydrazine, pH 8.0. The magnets were then removed and the separated material emerging from the column was collected. FIG. 1 shows Transmission Electron Micrographs (JEOL 2010) of the cobalt-platinum nanoparticles within apoferritins both (a) before and (b) after magnetic separation.

### Example 5

### Filtration of Nanoparticles

Suspensions of apoferritin, such as the material prepared in Example 1, or magnetic nanoparticles, such as the material prepared in Example 2, were subject to membrane filtration using Millipore® polysulphone filter having pore sizes ranging from 0.2 um-0.1 um. The eluates were collected and analysed by Transmission Electron Microscopy. Micrographs showed more than 70% of the particles in a composition as discrete individual particles.

1. A method for making a composition of magnetic nanoparticles which includes the step of forming said magnetic nanoparticles, each within a protein template, wherein a liquid composition of said protein templates or subunits thereof is subjected to a microporous membrane filtration step prior to formation of said magnetic nanoparticles.

2. A method in accordance with claim 1, wherein said protein template is selected from the group comprising flagellar L-P rings, microtubules, bacteriophages, chaperoning, virus capsids and members of the ferritin family.

3. A method in accordance with claim 2, wherein said protein template comprises a member of the ferritin family.

4. A method in accordance with claim 3, wherein said member of the ferritin family is selected from DPS and apoferritin.

5. A method in accordance with claim 4, wherein said member of the ferritin family is apoferritin.

6. A method in accordance with claim 1, wherein said magnetic nanoparticles comprise ferri- or ferro-magnetic metals, metal alloys, M-type or spinel ferrite.

7. A method in accordance with claim 6, wherein said ferri- or ferro-magnetic metal is selected from the group comprising cobalt, iron or nickel.

8. A method in accordance with claim 6, wherein said metal or metal alloy is selected from the group comprising aluminium, barium, bismuth, cerium, chromium, cobalt, copper, dysprosium, erbium, europium, gadolinium, holmium, iron, lanthanum, lutetium, manganese, molybdenum, neodymium, nickel, niobium, palladium, platinum, praseodymium, promethium, samarium, strontium, terbium, thulium, titanium, vanadium, ytterbium, and yttrium or a mixture thereof.

9. A method in accordance with claim 6, wherein said alloy is a binary alloy or a ternary alloy.

10. A method in accordance with claim 9, wherein said binary alloy is selected from the group comprising cobalt-nickel, iron-platinum, cobalt-palladium, iron-palladium, samarium-cobalt.

11. A method in accordance with claim 9, wherein said ternary alloy is selected from the group comprising dysprosium-iron-turbide or neodymium-iron boride, iron-cobalt-platinum, cobalt-nickel platinum, or cobalt-nickel-chromium.

12. A method in accordance with claim 1, wherein said nanoparticles comprise cobalt or platinum or alloys thereof.

13. A method in accordance with claim 12 wherein said nanoparticles comprise an alloy of cobalt and platinum.

14. A method in accordance with claim 1, wherein said liquid composition is an aqueous solution.

15. A method in accordance with claim 1, wherein the pore size of the membrane filter is in the range from about 0.02-10 μm.

16. A method in accordance with claim 1, wherein the pore size of the membrane filter is less than about 1 μm.

17. A method in accordance with claim 16, wherein the pore size of the membrane filter is less than about 0.5 μm.

18. A method in accordance with claim 17, wherein the pore size of the membrane filter is less than about 0.2 μm.

19. A method in accordance with claim 18, wherein the pore size of the membrane filter is about 0.1 μm.

20. A method in accordance with claim 1, wherein the membrane filter is a disc filter.

21. A method in accordance with claim 1, wherein the membrane filter is made from a material selected from the group comprising polymeric materials, metals, ceramics, glass or carbon.

22. A method in accordance with claim 21, wherein said material comprises a polymer.

23. A method in accordance with claim 22, wherein said polymer is selected from the group comprising polysulphones, polyethersulphones (PES), polyacrylates, polyvinylidenes, polytetrafluoroethylene (PTFE), cellulose, cellulose esters or co-polymers thereof.

24. A method in accordance with claim 23, wherein said polymer is polyethersulphone or a polyvinylidene.

25. A method in accordance with claim 1, wherein said protein is present in said liquid composition at a concentration in the range from about 10-50 mg/ml.

26. A method in accordance with claim 1, wherein the pH of said liquid composition is in the range from about 5.0-7.0.

27. A method in accordance with claim 1, wherein the liquid composition is subjected to an applied positive pressure during the filtration step.

28. A method in accordance with claim 1, wherein said magnetic nanoparticles have a diameter (or largest diameter in the case of non-spheroidal particles) not greater than about 100 nm.

29. A method in accordance with claim 28, wherein said magnetic nanoparticles have a diameter not greater than about 50 nm.

30. A method in accordance with claim 29, wherein said magnetic nanoparticles have a diameter of about 20 nm or less.

31. A method in accordance with claim 1, wherein said magnetic nanoparticles vary in their largest dimension by no more than about 20%.

32. A method in accordance with claim 31, wherein said magnetic nanoparticles vary in their largest dimension by no more than about 10%.

33. A method in accordance with claim 32, wherein said magnetic nanoparticles vary in their largest dimension by no more than about 5%.

34. A method in accordance with claim 1, wherein the formation step of said magnetic nanoparticles includes the incremental addition of sources of ions of the metal or metals to comprise or consist said magnetic nanoparticles.

35. A method in accordance with claim 34, wherein said cation and anion sources are added in sufficient amounts to provide 1-200 atoms of the cation and anion per encapsulating particle per iteration.

36. A method in accordance with claim 35, wherein said cation and anion sources are added in sufficient amounts to provide 20-100 atoms of the cation and anion per encapsulating particle per iteration.

37. A method in accordance with claim 36, wherein said cation and anion sources are added in sufficient amounts to provide about 50 atoms of the cation and anion per encapsulating particle per iteration.

38. A method in accordance with claim 34, wherein the source of the metal ions is a salt of the metal or metals.

39. A method in accordance with claim 38 wherein said salt is tetrachloroammoniumplatinate.

40. A method in accordance with claim 1, wherein the formation of said magnetic nanoparticles takes place under an inert atmosphere.

41. A method in accordance with claim 1, wherein the formation of said magnetic nanoparticles takes place at a temperature of at least about 24° C.

42. A method in accordance with claim 41, wherein the formation of said magnetic nanoparticles takes place at a temperature in the range from about 25° C. to about 60° C.

43. A method in accordance with claim 42, wherein the formation of said magnetic nanoparticles takes place at a temperature in the range from about 35° C. to about 50° C.

44. A method in accordance with claim 1, wherein the method further comprises a magnetic fractionation step.

45. A method in accordance with claim 44, wherein said magnetic fractionation step comprises passing said liquid composition through a column comprising magnetic powder at a flow rate in the range from about 0.2-10 ml/min⁻¹.

46. A method in accordance with claim 1, wherein the encapsulating shell is functionalised with a ligand selected from the group comprising biotin, avidin, an antibody or derivative thereof, a receptor molecule, an opsonin or a metal binding ligand.

47. A method in accordance with claim 1, wherein the encapsulating shell is removed or treated.

48. A method in accordance with claim 47, wherein said encapsulating shell is removed by enzymatic degradation or pH denaturation.

49. A method in accordance with claim 48, wherein the enzyme used to remove the protein is a protease.

50. A method in accordance with claim 48, wherein the pH denaturation is effected by adjusting the pH of the composition to a value below about 4.0 or above about 9.0.

51. A method in accordance with claim 47, wherein the encapsulating shell is carbonised.

52. A method for treating a liquid composition of magnetic nanoparticles, each formed within a macromolecular template, wherein said method includes the step of subjecting said composition to a microporous membrane filtration step.

53. A method in accordance with claim 52, wherein said macromolecular template comprises organic or inorganic material.

54. (canceled)

55. A method in accordance with claim 53, wherein said organic material is an organic macromolecule.

56. A method in accordance with claim 55, wherein said organic macromolecule is selected from the group comprising surfactants, polymers and proteins.

57.-60. (canceled)

61. A method in accordance with claim 52, wherein said magnetic nanoparticles comprise ferri- or ferro-magnetic metals, metal alloys, M-type or spinel ferrite.

62. A method in accordance with claim 61, wherein said ferri- or ferro-magnetic metal is selected from the group comprising cobalt, iron or nickel.

63. A method in accordance with claim 61, wherein said metal or metal alloy is selected from the group comprising aluminium, barium, bismuth, cerium, chromium, cobalt, copper, dysprosium, erbium, europium, gadolinium, holmium, iron, lanthanum, lutetium, manganese, molybdenum, neodymium, nickel, niobium, palladium, platinum, praseodymium, promethium, samarium, strontium, terbium, thulium, titanium, vanadium, ytterbium, and yttrium or a mixture thereof.

64.-68. (canceled)

69. A method in accordance with claim 52, wherein said liquid composition is an aqueous solution.

70. A method in accordance with claim 52, wherein the pore size of the membrane filter is in the range from about 0.02-10 μm.

71.-85. (canceled)

86. A method in accordance with claim 52, wherein said magnetic nanoparticles vary in their largest dimension by no more than about 20%.

87.-88. (canceled)

89. A method in accordance with claim 52, wherein the method further comprises a magnetic fractionation step.

90.-97. (canceled)

98. A stable composition of magnetic nanoparticles wherein each nanoparticle is encapsulated by an encapsulating material, wherein at least 70% by weight of the nanoparticles are not in an agglomerated form and wherein the composition comprises no more than 30% free encapsulating material, based on the total weight of the encapsulating material in the composition.

99.-108. (canceled)

109. A composition in accordance with claim 98, wherein said magnetic nanoparticles comprise ferri- or ferro-magnetic metals, metal alloys, M-type or spinel ferrite.

110. (canceled)

111. A composition in accordance with claim 109, wherein said metal or metal alloy is selected from the group comprising aluminium, barium, bismuth, cerium, chromium, cobalt, copper, dysprosium, erbium, europium, gadolinium, holmium, iron, lanthanum, lutetium, manganese, molybdenum, neodymium, nickel, niobium, palladium, platinum, praseodymium, promethium, samarium, strontium, terbium, thulium, titanium, vanadium, ytterbium, and yttrium or a mixture thereof.

112.-116. (canceled)

117. A composition in accordance with claim 98, wherein said composition is a liquid composition.

118.-119. (canceled)

120. A composition in accordance with claim 98, wherein said magnetic nanoparticles have a diameter (or largest diameter in the case of non-spheroidal particles) not greater than about 100 nm.

121.-122. (canceled)

123. A composition in accordance with claim 98, wherein said magnetic nanoparticles vary in their largest dimension by no more than about 20%.

124.-125. (canceled)

126. A composition in accordance with claim 98, wherein the encapsulating shell is functionalised with a ligand selected from the group comprising biotin, avidin, an antibody or derivative thereof, a receptor molecule, an opsonin or a metal binding ligand.

127.-131. (canceled)
